(19) United States
(12) Patent Application Publication
(10) Pub. No.: US 2025/0364154 A1
(43) Pub. Date: Nov. 27, 2025

(54) FLEXIBLE ELECTRODE APPARATUS FOR BONDING WITH SEEG ELECTRODE AND METHOD FOR MANUFACTURING SAME

(71) Applicant: CENTER FOR EXCELLENCE IN BRAIN SCIENCE AND INTELLIGENCE TECHNOLOGY, CHINESE ACADEMY OF SCIENCES, Shanghai (CN)

(72) Inventors: Zhengtuo ZHAO, Shanghai (CN); Xue LI; Chengcong FENG, Shanghai (CN); Jianing YANG, Shanghai (CN)

(21) Appl. No.: 18/875,083

(22) PCT Filed: Jun. 29, 2022
(86) PCT No.: PCT/CN2022/102151

§ 371 Date: Dec. 13, 2024

(30) Foreign Application Priority Data
Jun. 17, 2022 (CN) 202210689988.1

(51) Int. Cl. H01B 5/02; H01B 1/02; H01B 3/30; H01B 13/00; H01B 13/16; H01R 4/04; H01R 4/62

(52) U.S. Cl. CPC H01B 5/02; H01B 1/02; H01B 3/306; H01B 13/0013; H01B 13/0016; H01B 13/16; H01R 4/04; H01R 4/62; H01R 2201/12

(57) ABSTRACT

A flexible electrode apparatus for bonding with a SEEG electrode includes: at least one wire electrode which is implantable and flexible, wherein each wire electrode includes: a wire located between a first insulating layer and a second insulating layer of the flexible electrode; and an electrode site located on the second insulating layer and electrically coupled to the wire via a through hole in the second insulating layer, wherein the at least one wire electrode is configured to be affixed to the SEEG electrode and is in contact with a biological tissue after the SEEG electrode is implanted.

### TECHNICAL FIELD

The present disclosure relates to a flexible electrode apparatus for bonding with a stereotactic electroencephalography (SEEG) electrode and a method for manufacturing the same, and in particular, to a flexible electrode apparatus that can achieve firm attachment without using a specific adhesive and without significantly affecting the SEEG electrode, and a method for manufacturing the same.

### BACKGROUND

For patients with drug-resistant epilepsy, timely and correct diagnosis is conducive to doctors of different levels and epilepsy specialists to provide more effective treatment and service for patients. In the SEEG technology, a minimally invasive method is adopted without surgical incision, and only 2 mm micro-holes are drilled in a scalp and skull to place deep electrodes in specific locations deep in a brain. Therefore, this technology is suitable for epilepsy patients who need intracranial electrode electroencephalography (EEG) positioning.

In the SEEG technology, a positioning method is introduced from 2D level into 3D level, so that the electrode may be directly placed in the deep frontal lobe, medial side of the brain, cingulated gyrus, medial temporal lobe and other intracranial targeted part where conventional cortical electrodes cannot reach, so as to provide all-round stereo coverage of the brain, thereby achieving the purpose of accurately positioning the lesion and improving the treatment effect. The SEEG technology is a new epilepsy lesion positioning technology, which plays an important role in identifying lesions of epilepsy patients.

### SUMMARY

The present application proposes a flexible electrode apparatus for bonding with a SEEG electrode and a method for manufacturing the same.

According to a first aspect of embodiments of the present disclosure, a flexible electrode apparatus for bonding with a SEEG electrode is provided. The flexible electrode apparatus includes at least one wire electrode which is implantable and flexible, wherein each wire electrode includes: a wire located between a first insulating layer and a second insulating layer of the flexible electrode; and an electrode site located on the second insulating layer and electrically coupled to the wire via a through hole in the second insulating layer, wherein the at least one wire electrode is configured to be affixed to the SEEG electrode and is in contact with a biological tissue after the SEEG electrode is implanted.

According to a second aspect of embodiments of the present disclosure, a method for manufacturing a flexible electrode apparatus is provided, the flexible electrode apparatus including the flexible electrode for bonding with a SEEG electrode according to the first aspect. The method includes: forming a flexible separation layer over a substrate; forming the first insulating layer, a wire layer, the second insulating layer, and an electrode site layer over the flexible separation layer in a layer-by-layer manner; and removing the flexible separation layer to separate the flexible electrode from the substrate, wherein before the electrode site layer is formed, a through hole is formed at a position corresponding to the electrode site in the second insulating layer by patterning.

According to a third aspect of embodiments of the present disclosure, a method for processing a flexible electrode apparatus is provided, the flexible electrode apparatus including the flexible electrode for bonding with a SEEG electrode according to the first aspect. The method includes: causing the SEEG electrode to be in contact with and attached to a root portion of the flexible electrode in pure water; adjusting an attaching angle, and slowly pull an assembly of the SEEG electrode and the flexible electrode out of the water; and baking the assembly to enhance adhesion between the SEEG electrode and the flexible electrode.

Advantages of the embodiments according to the present disclosure lie in that, the flexible film and the SEEG electrode can be firmly attached without using any adhesive and without affecting the size, physicochemical properties and surgical process of the SEEG electrode, thereby providing a basis for surgical implantation of the SEEG electrode together with various flexible films, and expanding the application of the SEEG electrode. For example, the assembly of the SEEG electrode and the flexible electrode enables the SEEG electrode to have functions such as multi-channel, single-cell-level precise EEG signal acquisition and electrical stimulation.

It should be appreciated that the above advantages are not required to be all realized in one or some specific embodiments, but may be partially distributed in different embodiments according to the present disclosure. The embodiments according to the present disclosure may have one or some of the above advantages, or may have other advantages alternatively or additionally.

Other features and advantages of the present invention will become more apparent from the following detailed description of exemplary embodiments of the present invention with reference to the accompanying drawings.

### BRIEF DESCRIPTION OF THE DRAWINGS

FIG. 1 is a schematic exploded view showing a flexible electrode according to an embodiment of the present disclosure.

FIG. 2 is a schematic diagram showing a flexible electrode apparatus bonded with a SEEG electrode according to an embodiment of the present disclosure from different perspectives.

FIG. 3 is a schematic diagram showing an end portion of a flexible electrode apparatus bonded with a SEEG electrode according to an embodiment of the present disclosure.

FIG. 4 is another schematic diagram showing a flexible electrode apparatus bonded with a SEEG electrode according to an embodiment of the present disclosure.

FIG. 5 is a flowchart showing a method of manufacturing a flexible electrode according to an embodiment of the present disclosure.

FIG. 6 is a schematic diagram showing a method of manufacturing a flexible electrode according to an embodiment of the present disclosure.

FIG. 7 is a schematic diagram showing a method of affixing a flexible electrode to a SEEG electrode according to an embodiment of the present disclosure.

FIG. 8 is a flowchart showing a method of affixing a flexible electrode to a SEEG electrode according to an embodiment of the present disclosure.

### DETAILED DESCRIPTION

Various exemplary embodiments of the present disclosure will be described in detail below with reference to the accompanying drawings. It should be noted that unless otherwise specifically stated, the relative arrangement of components and steps, numerical expressions and numerical values set forth in these embodiments do not limit the scope of the present disclosure.

The following description of at least one exemplary embodiment is in fact merely illustrative and is in no way intended to limit the present disclosure and its application or use. That is, the structures and methods herein are shown in an exemplary manner to illustrate different embodiments of the structures and methods in the present disclosure. However, those skilled in the art will appreciate that they merely illustrate exemplary ways of the present disclosure that can be implemented, rather than exhaustive ways. In addition, the accompanying drawings need not be drawn to scale, and some features may be enlarged to illustrate the details of specific components.

Technologies, methods, and devices known to those of ordinary skills in the relevant art may not be discussed in detail, but where appropriate, such technologies, methods, and devices should be considered as a part of the granted specification.

The inventors of this application found in their research that the existing SEEG technology is limited by volume, channel count, and electrode site size. Even if it can be used to locate epileptic lesions, it is not possible to perform accurate EEG data acquisition, electrical stimulation, and monitoring of the microenvironment in the brain at the single-cell level. Specifically, being limited to the volume and original design, a single SEEG electrode generally has a channel count of a dozen or so and large electrode sites at millimeter scale. Therefore, the channel count is low, the large amount of channel information is complicated, and the recording accuracy is low. The data usually obtained is a local field potential signal (LFP), which is not competent for EEG signal acquisition at the single-cell level; and the SEEG electrode has single function usually for positioning epileptic lesions, while lacking extendibility to other functions. When the SEEG electrode is used in combination with the flexible electrode, it may use the flexible electrode to record spike data, so as to improve the precision and accuracy of epileptic lesion positioning, and also provide other medical or scientific research use.

Based on this, in the technical solution of the present application, an attempt is made to attach an ultra-thin and ultra-flexible film electrode to the SEEG electrode to improve and expand the function of the SEEG electrode.

In summary, the technical solution of the present disclosure mainly relates to a flexible electrode for electrical stimulation and electrical signal acquisition of a brain, which has technical effects such as smaller size, better adhesion and multi-channel, and the flexible electrode is bonded with a SEEG electrode to be implanted into a brain area together, so as to obtain expanded comprehensive detection results, such as realizing multi-channel, single-cell-level precise EEG signal acquisition and electrical stimulation, and physiological signal monitoring (ion concentration, pH value, etc.), etc.

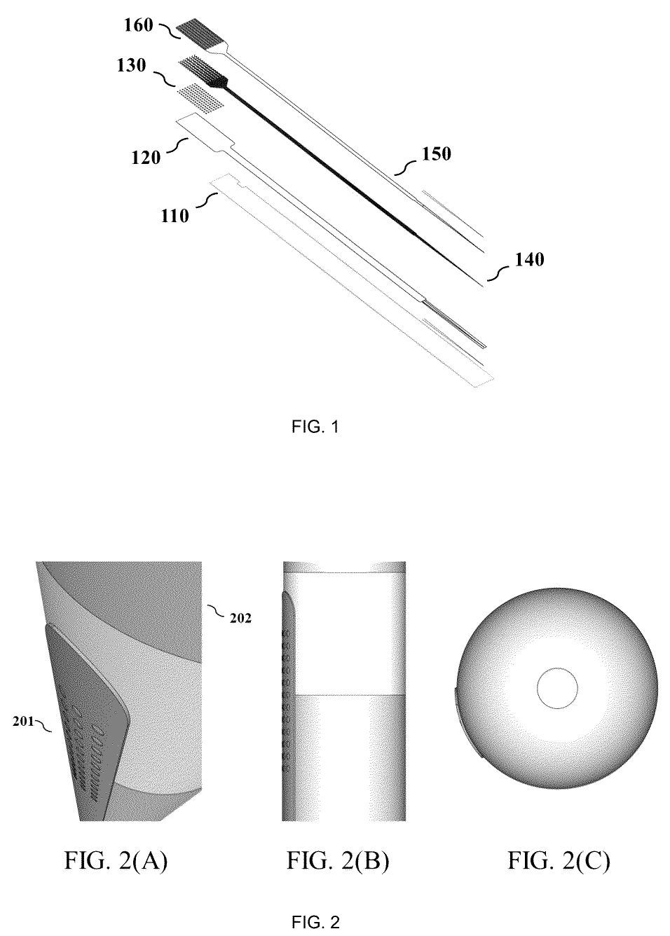

FIG. 1 shows an exploded view of a flexible electrode according to an embodiment of the present disclosure. As shown in FIG. 1, the flexible electrode may have a strip-like shape, and may include a wire portion to be connected to an external circuit, an electrode site, an attachment portion (back-end portion) to be attached to a SEEG electrode, and a contact portion to be in contact with a biological tissue, etc. It should be noted that the actual shape and/or individual components of the electrode may be designed as needed, instead of being limited to the shown shapes and size relationships. Specifically, it can be clearly seen from the figure that the flexible electrode has a multi-layer structure, which specifically includes a flexible separation layer 110, a first insulating layer 120, a circuit board connection layer 130, a wire layer 140, a second insulating layer 150, and an electrode site layer 160, etc. It should be understood that the distribution of the layers of the flexible electrode shown in FIG. 1 is only a non-limiting example, and the flexible electrode in the present disclosure may not include one or more of these layers, or may include more other layers.

As shown in FIG. 1, wires in the flexible electrode include a plurality of wires located in the wire layer and spaced apart from each other, wherein electrode sites in the flexible electrode include a plurality of electrode sites each electrically coupled to one of the plurality of wires via a corresponding through hole in the bottom insulating layer. The flexible electrode has good flexibility, and can be partially or completely implantable into the biological tissue to acquire or apply electrical signals from or to the biological tissue. The conductive layer of the flexible electrode shown in FIG. 1 includes a plurality of wires, but it should be understood that in different embodiments, the electrode in the present disclosure may include a single wire or other specified number of wires. These wires may have a width and thickness at the nanometer level or micrometer level, and a length that is several orders of magnitude larger than the width and thickness as required (such as at the centimeter level). In the embodiments according to the present disclosure, the shape, size, etc. of these wires are not limited to the ranges listed above, but may vary according to design requirements.

Specifically, the flexible electrode may include the first insulating layer 120 located at the bottom of the electrode and the second insulating layer 150 located at the top of the electrode. The insulating layer in the flexible electrode may refer to an outer surface layer of the electrode that plays an insulating role. Since the insulating layer of the flexible electrode needs to be in contact with the biological tissue after implantation, the material of the insulating layer is required to have good biocompatibility while having good insulation. In an embodiment of the present disclosure, the material of the insulating layers 120 and 150 may include polyimide (PI), polydimethylsiloxane (PDMS), parylene, epoxy resin, or polyamide imide (PAI), etc. In addition, the insulating layers 120 and 150 are also main parts of the flexible electrode that provide strength. If the insulating layer is too thin, the strength of the electrode will be reduced; and if the insulating layer is too thick, the flexibility of the electrode will be reduced, and the implantation of an electrode including an overly thick insulating layer will cause greater damage to the organism. In an embodiment according to the present disclosure, the thickness of the insulating layers 120, 150 may be 100 nm to 300 μm, preferably 300 nm to 3 μm, more preferably 1 μm to 2 μm, or 500 nm to 1 μm, etc.

The wire layers in the flexible electrode are distributed in the wire layer 140 between the first insulating layer 120 and the second insulating layer 150. In an embodiment according to the present disclosure, each flexible electrode may include one or more wires located in the same wire layer 140. For example, it can be clearly seen from FIG. 1 that the wire layer 140 of the flexible electrode includes a plurality of wires, each of which includes an elongated main body portion and an end portion corresponding to a corresponding electrode site. The line width of the wire and the spacing between the wires may be, for example, 10 nm to 500 μm. The spacing between the wires may be, for example, as low as 10 nm, for example, preferably 100 nm to 3 μm. It should be understood that the shape, size, spacing, etc. of the wires are not limited to the ranges listed above, but may vary according to design requirements.

In an embodiment according to the present disclosure, the wire in the wire layer 140 may have a thin film structure including multiple sub-layers stacked in the thickness direction. The materials of these sub-layers may be materials that can enhance properties such as adhesion, ductility, and conductivity of the wire. As a non-limiting example, the wire layer 140 may include a conductive sub-layer and an adhesion sub-layer that are stacked, wherein the adhesion sub-layer that is in contact with the insulating layer 120 and/or 150 is made of a metal adhesion material such as titanium (Ti), titanium nitride (TiN), chromium (Cr), tantalum (Ta), or tantalum nitride (TaN), or a non-metal adhesion material; and the conductive sub-layer is made of a material with good conductivity such as gold (Au), platinum (Pt), iridium (Ir), tungsten (W), magnesium (Mg), molybdenum (Mo), platinum-iridium alloy, titanium alloy, graphite, carbon nanotubes, or PEDOT, etc. It should be understood that the wire layer may also be made of other metal or non-metal materials that have conductivity, or may be made of conductive polymer materials and conductive composite materials. In a non-limiting embodiment, the conductive sub-layers of these wires have a thickness of 5 nm to 200 μm, and their adhesion sub-layers have a thickness of 1 to 50 nm.

The flexible electrode may further include an electrode site in the electrode site layer 160 located above the first insulating layer 120, and the electrode site may be in contact with the biological tissue to directly acquire or apply an electrical signal after the flexible electrode is implanted. In the flexible electrode, the electrode site in the electrode site layer 160 may be electrically coupled to the corresponding wire via a through hole at a position corresponding to the electrode site in the first insulating layer 120. In the case where the flexible electrode includes a plurality of wires, the flexible electrode may correspondingly include a plurality of electrode sites in the electrode site layer 160, and each of the electrode sites may be electrically coupled to one of the plurality of wires via a corresponding through hole in the first insulating layer 120.

In a non-limiting embodiment, each electrode site may correspond to a wire in the wire layer 140. Each electrode site may have a planar size at the micrometer level and a thickness at the nanometer level. In an embodiment according to the present disclosure, the electrode sites may include sites with a diameter of 1 μm to 500 μm, and the spacing between the electrode sites may be 1 μm to 5 mm. In an embodiment according to the present disclosure, the electrode sites may have a shape of a circle, an ellipse, a rectangle, a rounded rectangle, or a chamfered rectangle, etc. It should be understood that the shape, size, spacing, etc. of the electrode sites may be selected according to the situation of the biological tissue area required to be recorded.

In an embodiment according to the present disclosure, the electrode site in the electrode site layer 160 may have a thin film structure including multiple sub-layers stacked in the thickness direction. The material of a sub-layer close to the wire layer 140 among the multiple sub-layers may be a material that can enhance the adhesion between the electrode site and the wire. As a non-limiting example, the electrode site layer 160 may be a metal film including two superimposed sub-layers, wherein a first sub-layer close to the wire layer 140 is made of Ti, TiN, Cr, Ta or TaN, and a second sub-layer of the electrode site layer 260 that is exposed to the outside is made of Au. It should be understood that the electrode site layer may also be similar to the wire layer, and may be made of other metal or non-metallic materials that have conductivity, such as Pt, Ir, W, Mg, Mo, platinum-iridium alloy, titanium alloy, graphite, carbon nanotubes, or PEDOT, etc.

In an embodiment according to the present disclosure, a surface of the electrode site that is exposed to the outside and in contact with the biological tissue may further have a surface modification layer to improve the electrochemical properties of the electrode site. As a non-limiting example, the surface modification layer may be obtained by using an electro-induced polymerization coating of PEDOT:PSS, sputtering an iridium oxide film, and the like, and is used to reduce impedance (such as electrochemical impedance at an operating frequency of 1 kHz) in a case where the flexible electrode acquires electrical signals, and to improve charge injection capability in a case where the flexible electrode applies electrical signals for stimulation, thereby improving interaction efficiency.

In an embodiment according to the present disclosure, the flexible electrode may further include a bottom electrode site layer (not shown) located below the first insulating layer 120, and the electrode site therein may be in contact with a biological tissue to directly acquire or apply an electrical signal after the flexible electrode is implanted. Specifically, the electrode site in the bottom electrode site layer is similar to the electrode site in the electrode site layer 160, and in the flexible electrode, the electrode site in the bottom electrode site layer may be electrically coupled to a corresponding wire via a through hole at a position corresponding to the electrode site in the bottom insulating layer. In an embodiment according to the present disclosure, the electrode site in the bottom electrode site layer and the electrode site in the electrode site layer 160 may be located at opposite positions on the top and bottom sides of the flexible electrode, and may be electrically coupled to the same wire in the wire layer 140. In an embodiment according to the present disclosure, the electrode site in the bottom electrode site layer and the electrode site in the electrode site layer 160 may also be located at different positions on the top and bottom sides of the flexible electrode to acquire or apply electrical signals in different areas of a biological tissue; and in an embodiment according to the present disclosure, the electrode site in the bottom electrode site layer and the electrode site in the electrode site layer 160 may also be electrically coupled to different wires in the wire layer 140.

In an embodiment according to the present disclosure, the flexible electrode may further include the flexible separation layer 110. The flexible separation layer 110 is mainly used in the manufacturing process of the flexible electrode. The flexible separation layer can be removed by a specific substance to separate a part of the flexible electrode and avoid damage to the flexible electrode, and is provided with an adhesion layer. The material of the flexible separation layer is any one of nickel, chromium, or aluminum, or a combination thereof. The flexible separation layer 110 is further provided with an adhesion layer made of a material including chromium, tantalum, tantalum nitride, titanium, or titanium nitride.

It should be understood that the bottom electrode site layer is an optional part but not a necessary part of the flexible electrode. For example, in the exploded structure shown in FIG. 1, the flexible electrode may only include the electrode site layer 160 and may not include the bottom electrode site layer. The shape, size, material, etc. of the bottom electrode site may be similar to those of the top electrode site, and will not be described in detail here.

In an embodiment according to the present disclosure, the back-end portion of the flexible electrode may include at least one back-end site, and each of the attachment portions of the flexible electrode attached to an optical device extends from the back-end portion; and the back-end site may be electrically coupled to a back-end circuit and one of the wires via through hole(s) in the first insulating layer 120 and/or the second insulating layer 150 to achieve bidirectional signal transmission between the back-end circuit and the electrode site electrically coupled to the wire. Here, the back-end circuit may refer to a circuit at the back end of the flexible electrode, such as a recording circuit, or a processing circuit, etc. associated with the signal of the flexible electrode. In an embodiment according to the present disclosure, the flexible electrode may be coupled to the back-end circuit in a connection manner. Specifically, a ball grid array (BGA) package site as the back-end site may be adaptedly connected to a commercial signal recording system through a printed circuit board (PCB), a flexible printed circuit (FPC), etc., and the connection manner includes solder ball attachment and anisotropic conductive film bonding (ACF Bonding), etc. In an embodiment according to the present disclosure, the flexible electrode may also be integrated with the back-end circuit. Specifically, pre-processing functions such as signal amplification and filtering may be integrated on a dedicated chip, and then the chip is connected and packaged with the integrated PCB at the back end of the flexible electrode by bonding or other methods, so as to achieve wireless transmission and charging, etc. In this case, an independent flexible electrode and an independent dedicated chip as the back-end circuit may be used, and the electrical connection between the flexible electrode and the dedicated chip may be made by solder ball attachment or ACF Bonding or other methods. Alternatively, a certain space may be reserved on a wafer of a chip as the back-end circuit, for which tape-out has been done in advance, and the electrode may be directly manufactured on this basis, thereby realizing the joint processing or separate processing of the chip and the electrode to achieve a higher level of integration.

The back-end site may have a planar size at the micrometer level and a thickness at the nanometer level. As a non-limiting example, the back-end site may be the BGA package site with a diameter of 50 μm to 2000 μm, or may be a circular, elliptical, rectangular, rounded rectangular, or chamfered rectangular site with a side length of 50 μm to 2000 μm. It should be understood that the shape, size, etc. of the back-end sites are not limited to the ranges listed above, but may vary according to design requirements.

The back-end site for connection may include multiple sub-layers in the thickness direction, the material of an adhesion sub-layer close to the wire layer 140 among the multiple sub-layers may be a material that can enhance the adhesion between the electrode site and the wire; the material of a flux sub-layer in the middle among the multiple sub-layers may be a flux material; the material of a conductive sub-layer among the multiple sub-layers may be above-described other conductive metal materials or conductive non-metal materials of the wire layer 140; and the outermost layer of the multiple sub-layers that may be exposed through the insulating layers 120 and 150 is an anti-oxidation protective sub-layer. As a non-limiting example, the back-end site layer may include a conductive sub-layer and an adhesion sub-layer that are stacked, wherein the adhesion sub-layer close to the wire layer 140 may be a nano-scale sub-layer to improve the adhesion between the back-end site layer and the wire layer 140, an adhesion layer as the middle flux sub-layer may be made of nickel (Ni), Pt or palladium (Pd), and a third sub-layer as the outermost conductive sub-layer may be made of Au, Pt, Ir, W, Mg, Mo, platinum-iridium alloy, titanium alloy, graphite, carbon nanotubes, or PEDOT, etc. It should be understood that the back-end site layer may also be made of other conductive metal materials or conductive non-metallic materials. The back-end site layer in FIG. 1 is a part connected to a back-end processing system or chip, and the design of the size, spacing, shape, etc. of the sites therein may be changed according to different connection manner at the back-end. In a non-limiting embodiment, the flexible electrode adopted has electrode sites with 512 channels, including four 128 BGAs. It should be understood that electrode sites with other channel counts may be included as needed, such as 32, 36, 64, 128 channels, etc.

In an embodiment according to the present disclosure, the flexible electrode may not include site layers such as the electrode site layer (and/or the bottom electrode site layer), or the back-end site layer, etc. In this case, the electrode sites and the back-end sites used for adaptation in the back-end portion of the flexible electrode may both be parts of the wire layer, and electrically coupled to the corresponding wires in the wire layer. In addition, the electrode sites for sensing and applying electrical signals may be in direct contact with the tissue area into which the wire electrode is implanted. As a non-limiting example, each electrode site may be electrically coupled in the wire layer to a corresponding wire in the wire layer, and exposed at the outer surface of the wire electrode via a corresponding through hole in the top insulating layer or the bottom insulating layer and in contact with the biological tissue.

FIG. 2 is a schematic diagram showing an apparatus obtained after the flexible electrode according to the present disclosure is bonded with the SEEG electrode from different perspectives, wherein (A) to (C) respectively show a state of the flexible electrode after being bonded with the SEEG electrode from the lateral upper side, the side surface, and the top surface. In a non-limiting embodiment, as shown in the views, a SEEG electrode 201 has a shape of a roughly long cylinder, and a flexible electrode 202 is attached to the cylindrical outer wall of the SEEG electrode 201.

As shown in FIG. 2, the inner diameter of the SEEG electrode is typically 0.5 mm to 2 mm, preferably 1 mm as shown. The commonly used thickness for the flexible electrode is 300 nm to 10 μm, and the thickness as shown is 10 μm; and the commonly used width for the flexible electrode is 100 μm to 500 μm, and the specific width may be adjusted according to the usage scenario and function. In addition, it is possible to achieve tight attachment between the flexible electrode and the SEEG electrode without any adhesive, which will be described later. Since the flexible electrode itself is ultra-thin and ultra-flexible and has good attachability, and the size and position of the flexible electrode relative to the SSEG electrode may be adjusted in practical application, the size (such as cross-sectional area), physicochemical properties and/or implantation surgical process of the SEEG electrode will not be significantly affected after the SEEG electrode is assembled with the flexible electrode.

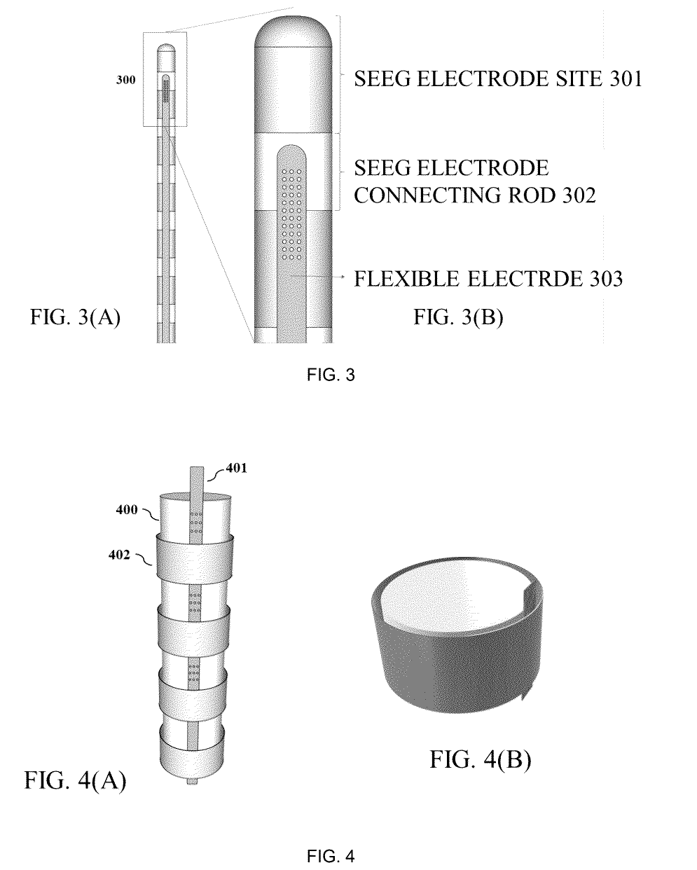

The above-mentioned apparatus obtained after the flexible electrode is bonded with the SEEG electrode is further shown in FIG. 3, wherein FIG. 3(A) shows a front schematic diagram of the apparatus, and FIG. 3(B) is an enlarged schematic diagram of an area 300 in (A) (i.e., an end portion of the apparatus). In a non-limiting embodiment, as shown, a SEEG electrode site 301 is made of a metal material, such as any one of platinum-iridium alloy, platinum, silver, or stainless steel, or a combination thereof, and a SEEG electrode connecting rod 302 between the electrode sites is generally made of an insulating material. The electrode sites of the flexible electrode 303 are partially attached to the outer side wall of the electrode connecting rod 302 to form a relatively tightly attached assembly.

Alternatively, FIG. 4 is another schematic diagram showing a flexible electrode apparatus bonded with a SEEG electrode according to an embodiment of the present disclosure. That is, in addition to the attachability of the flexible electrode itself, the flexible electrode may also be connected mechanically. Specifically, as shown in FIG. 4, the structure of the SEEG electrode 400 may be customized so that SEEG electrode sites 402 (usually having a metal ring structure) or the connecting rods form gaps through which a flexible electrode 401 may pass. FIG. 4(A) and (B) respectively show the metal ring of the customized electrode site 401 and its enlarged schematic diagram. It should be noted that the gap in FIG. 4(B) is only schematic, and the size relationship between the gap size and the diameter of the SSEG electrode in actual application is not the case. In addition, after the flexible electrode 402 passes through the gap of the metal ring of the electrode site 401, a tight connection between the flexible electrode and the SEEG electrode is formed by methods such as thermal contraction or thermal expansion. Alternatively, a groove adapted to the shape of the flexible electrode 401 may be customized on the structure of the SEEG electrode 400, so that the flexible electrode 401 is fixed in the groove, and the flexible electrode 401 and the SEEG electrode 400 will not move relative to each other unexpectedly during the implantation process.

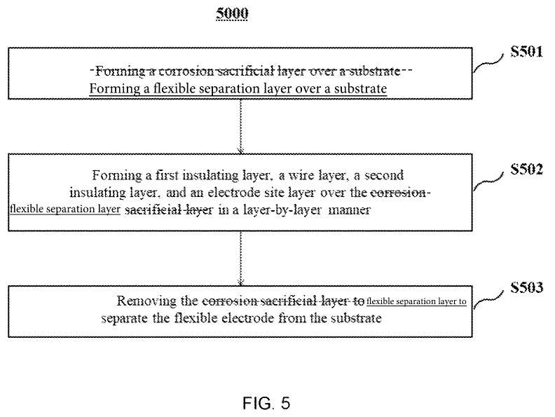

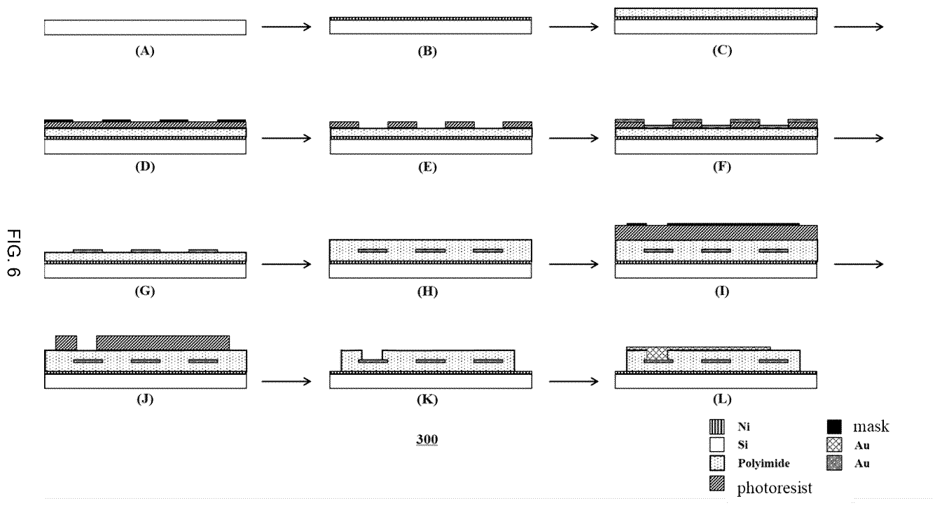

FIG. 5 is a flowchart showing a method for manufacturing a flexible electrode according to an embodiment of the present disclosure. In the present disclosure, a manufacturing method based on a micro-electro mechanical system (MEMS) process may be adopted to manufacture a nano-scale flexible electrode. As shown in FIG. 5, the method 5000 may include: at S501, forming a flexible separation layer over a substrate; at S502, forming a first insulating layer, a wire layer, a second insulating layer, and an electrode site layer over the flexible separation layer in a layer-by-layer manner, wherein before an electrode site is formed, a through hole is formed at a position corresponding to the electrode site in the first insulating layer by patterning; and at S503, removing the flexible separation layer to separate the flexible electrode from the substrate.

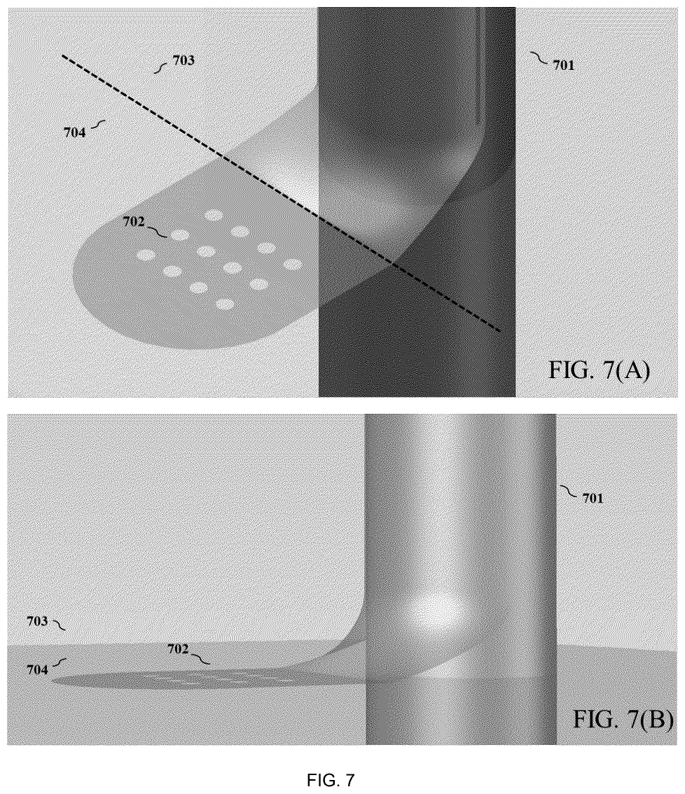

FIG. 6 shows a schematic diagram of a method for manufacturing a flexible electrode according to an embodiment of the present disclosure. The forming process and structures of the flexible separation layer, bottom insulating layer, wire layer, top insulating layer, electrode site layer, etc., of the flexible electrode are described in more detail in conjunction with FIG. 6.

The view (A) of FIG. 6 shows a substrate of the electrode. In an embodiment according to the present disclosure, a hard substrate such as glass, quartz, silicon wafer, etc., may be used. In an embodiment according to the present disclosure, other soft materials may also be used as the substrate, such as the same material as that of the insulating layer.

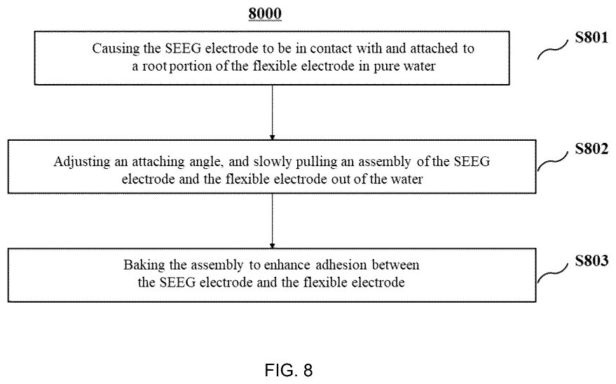

The view (B) of FIG. 6 shows a step of forming a flexible separation layer on the substrate. The flexible separation layer may be removed by applying a specific substance, so as to facilitate the separation of the flexible portion of the electrode from the hard substrate. In the embodiment shown in FIG. 8, Ni is used as the material of the flexible separation layer, and other materials such as Cr and Al may also be used. In an embodiment according to the present disclosure, when a flexible separation layer is formed on the substrate by evaporation, a portion of the exposed substrate may be etched first to improve the flatness of the entire substrate after evaporation. It should be understood that the flexible separation layer is an optional part but not a necessary part of the flexible electrode. Depending on the characteristics of the selected material, the flexible electrode may also be conveniently separated without the flexible separation layer. In an embodiment according to the present disclosure, the flexible separation layer may also be provided with a mark which may be used for alignment of subsequent layers.

The view (C) of FIG. 6 shows forming a bottom insulating layer on the flexible separation layer. As a non-limiting example, in the case where the insulating layer is made of the polyimide material, the forming of the bottom insulating layer may include steps such as a film forming process, a formed-film curing, and an enhanced curing to form a thin film as the insulating layer. The film forming process may include applying polyimide on the flexible separation layer, for example, a layer of polyimide may be formed by spin coating in segmented rotational speeds. The formed-film curing may include gradually heating to a high temperature and keeping the temperature to form a film, so as to perform subsequent processing steps. The enhanced curing may include multi-gradient temperature increasing before forming subsequent layers, preferably in a vacuum or nitrogen atmosphere, and baking for several hours. It should be understood that the above forming process is only a non-limiting example of the forming process of the bottom insulating layer, and one or more of the steps may be omitted, or more other steps may be included.

It should be noted that the above forming process is directed to an embodiment where a bottom insulating layer is formed in a flexible electrode without a bottom electrode site layer and there is no through hole corresponding to the electrode sites in the bottom insulating layer. If the flexible electrode includes a bottom electrode site layer, the bottom electrode site layer may be formed on the flexible separation layer before forming the bottom insulating layer. For example, Au and Ti may be sequentially evaporated on the flexible separation layer. The step of patterning the bottom electrode sites will be described in detail later with respect to the top electrode sites. Accordingly, in the case where the flexible electrode includes bottom electrode sites, in the process of forming the bottom insulating layer, in addition to the above steps, a patterning step may also be included for etching through holes at positions corresponding to the bottom electrode sites in the bottom insulating layer. The step of patterning the insulating layer will be described in detail later with respect to the top insulating layer.

Views (D) to (G) of FIG. 6 show forming a wire layer on the bottom insulating layer. As shown in the view (D), a photoresist and a mask may be applied on the bottom insulating layer. It should be understood that other photolithography methods may also be used to prepare the patterned thin film, such as laser direct writing and electron beam photolithography. In an embodiment according to the present disclosure, for a metal film such as the wire layer, a double-layer photoresist may be applied to facilitate the forming (evaporation or sputtering) and stripping of the patterned thin film. By setting the pattern of the mask associated with the wire layer, for example, the pattern of the wire layer 140 shown in FIG. 1 may be implemented, that is, the contour of one or more wires in the wire electrodes extending from the back-end portion. Then, exposure and development may be performed to obtain a structure as shown in the view (E). In an embodiment according to the present disclosure, the exposure may be carried out by contact photolithography, and the mask and the structure are exposed in a vacuum contact mode. In an embodiment according to the present disclosure, different developers and concentrations thereof may be adopted for patterns of different sizes. This step may also include alignment between the layers. Next, a film may be formed on the structure shown in the view (E), for example, processes such as evaporation and sputtering may be used to deposit a metal thin film material, such as Au, to obtain a structure shown in the view (F). Next, stripping may be performed to separate the thin film in the non-patterned area from the thin film in the patterned area by removing the photoresist in the non-patterned area, so as to obtain a structure as shown in the view (G), that is, to form a wire layer. In an embodiment according to the present disclosure, a photoresist removal may be performed again after the photoresist stripping, so as to further remove the residual photoresist on the surface of the structure.

In an embodiment according to the present disclosure, a back-end site layer may also be formed before forming the wire layer. As a non-limiting example, the forming process of the back-end site layer may be similar to that of the metal film described above with respect to the wire layer.

Views (H) to (K) of FIG. 6 show forming a top insulating layer. For photosensitive thin films, patterning may be generally achieved directly through patterned exposure and development, while for non-photosensitive materials used in the insulating layer, patterning cannot be achieved by exposing and developing the materials themselves. In this case, a sufficiently thick patterned anti-etching layer may be formed on this layer, and then the thin film in an area not covered by the anti-etching layer is removed by dry etching (the anti-etching layer will also be thinned at the same time, so it is necessary to ensure that the anti-etching layer is thick enough), and then the anti-etching layer is removed to achieve the patterning of the non-photosensitive layer. As a non-limiting example, when forming the insulating layer, the photoresist may be used as the anti-etching layer. The forming of the top insulating layer may include steps such as a film forming process, formed-film curing, patterning, and enhanced curing, wherein the view (H) shows a structure obtained after the film-forming of the top insulating layer, the view (I) shows the application of photoresist and mask on the top insulating layer after the film-forming, the view (J) shows a structure including the anti-etching layer obtained after exposure and development, and the view (K) shows a structure including the top insulating layer formed. The film forming process, formed-film curing and enhanced curing have been described in detail above with respect to the bottom insulating layer, and description therefor is omitted here for brevity. The patterning step may be performed after the formed-film curing, or after the enhanced curing. After the enhanced curing, the insulating layer has a stronger anti-etching ability. Specifically, in the view (I), a sufficiently thick layer of photoresist is formed on the insulating layer through steps such as spin coating and baking. By providing the pattern of the mask related to the top insulating layer, for example, the pattern of the top insulating layer shown in FIG. 1 may be realized, that is, the contour of the top insulating layer implemented on the one or more wires in the wire electrodes extending from the back-end portion and the contours of the through holes implemented at positions corresponding to the electrode sites in the top insulating layer. In the view (J), the pattern is transferred to the photoresist on the insulating layer through steps such as exposure and development to obtain an anti-etching layer, wherein the portion to be removed from the top insulating layer is exposed. The exposed portion of the top insulating layer may be removed by oxygen plasma etching, to obtain a structure shown in the view (K).

In the embodiment according to the present disclosure, an adhesion enhancement treatment may be performed before forming the top insulating layer, so as to improve the bonding force between the bottom insulating layer and the top insulating layer.

The view (L) of FIG. 6 shows forming a top electrode site layer on the top insulating layer by evaporation or the like.

Next, a method of affixing the flexible electrode to the SEEG electrode according to an embodiment of the present disclosure will be described in conjunction with FIG. 7.

Generally, when the flexible electrode according to the present disclosure is attached to the SEEG electrode, multiple forces will be formed between the two electrodes at the same time, and a resultant force of these forces creates technical effects of close attachment and difficulty of peeling. These forces include but are not limited to the following:
(1) mechanical bonding: it is commonly seen as the adhesive force between the hot melt adhesive film and an adherend; and the friction force generated between the flexible electrode and the SEEG electrode after drying and curing forms a mechanical bonding force.
(2) van der Waals force: when two materials approach so that a distance therebetween is small enough, the van der Waals force or hydrogen bonding occurs between molecules, thus obtaining good adhesion; and the materials of the SEEG electrode connecting rods and the flexible film are both non-polar materials, which make it easy to form this kind of molecular force.
(3) mutual diffusion: the adhesion between polymer compounds is formed by the diffusion caused by the thermal motion of the macromolecules themselves or their chain segments, which is in essence mutual dissolution that occurs at the interface, thus forming a strong bond.
(4) charge attraction: it comes from the attraction between positive and negative charges in the electric double layer, and this attraction is proportional to the square of the charge density.

The above embodiments respectively illustrate the common manifestations of the above-mentioned forces. The apparatuses in FIGS. 2 and 3 mainly show examples of attaching the flexible electrode to the surface of the SEEG electrode, which achieves the attachment between the flexible electrode and the SEEG electrode without using any adhesive and without relying on the constraints of mechanical structures. Alternatively, the apparatus in FIG. 4 shows another example of using mechanical structures to assist the affixing of the electrodes, in which a gap, through which the flexible electrode can pass, is formed by customizing the structure of the SEEG electrode.

In a non-limiting embodiment, the method of attaching the flexible electrode to the SEEG electrode requires at least pure water (distilled water level or higher), an open container that can be used to hold the water (including but not limited to a beaker, or a petri dish, etc.), tools required to guide the flexible film (including but not limited to a thin tungsten wire, a toothpick, or a syringe needle, etc.), and/or auxiliary tools such as an oven and a high-temperature resistant container (including but not limited to a glass petri dish, an enamel tank, etc.).

Experiments show that the flexible electrode is usually made of non-polar materials. If an end of the electrode that will first contact a brain surface or other substance (such as water) is affixed to a metal SEEG electrode site made of a polar material, it will easily detach due to insufficient bonding force. Therefore, it is recommended to bond the end that will first enter the brain area to a SEEG connecting rod made of a non-polar material.

Specifically, FIG. 7 shows a SEEG electrode 701 and a flexible electrode 702 that are being attached, and FIG. 7 (A) and (B) respectively show air 703, liquid (such as pure water) 704 and a boundary therebetween from different perspectives. A root portion of the flexible electrode 702 is in contact with and attached to the SEEG electrode 701 in the pure water, and after an attaching angle has been adjusted, the resulted assembly is slowly pulled out of the water along a direction indicated by an arrow in FIG. 7. At this time, the remaining part of the flexible electrode 702 will be attached to the surface of the SEEG electrode 701 in sequence under the surface tension of the water, so that the end portion of the flexible electrode 702 is finally placed on the non-metallic part of the SEEG electrode 701. After an apparatus obtained after the SEEG electrode 701 is bonded with the flexible electrode 702 is completely lifted out of the water, the apparatus is baked to enhance the adhesion between the SEEG electrode 701 and the flexible electrode 702.

In a non-limiting embodiment, the apparatus obtained after the SEEG electrode 701 is bonded with the flexible electrode 702 is placed in a high-temperature resistant container and then put into an oven. The high-temperature resistant container is preferably provided with a lid to prevent air flow interference in the oven. The temperature and duration for baking in the oven depend on the high-temperature resistance of the flexible electrode 702 and the SEEG electrode 701. It is generally believed that the temperature should be above 40° C., preferably 60° C. to 200° C., and the duration should be more than 3 minutes.

It should be noted that the most advantageous technical effect of the technical solution of the present application lies in that the flexible electrode is affixed to the SEEG electrode without any adhesive. Alternatively, the flexible electrode may be attached to the SEEG electrode by a variety of adhesives including biodegradable materials such as polyethylene glycol, polylactic acid, polylactic acid-glycolic acid copolymer, or silk protein, etc.

FIG. 8 is a flowchart showing a method for affixing a flexible electrode to a SEEG electrode according to the aforementioned embodiment. Specifically, in step S801, the SEEG electrode is caused to be in contact with and attached to the root portion of the flexible electrode in pure water. Then, in step S802, the attaching angle is adjusted, and the assembly of the SEEG electrode and the flexible electrode is slowly pulled out of the water. Finally, at step S803, the assembly is baked to enhance the adhesion between the SEEG electrode and the flexible electrode.

Alternatively, the technical solution of the present application can also be used in other application scenarios. The flexible electrode according to the present disclosure can be used in combination with a DBS electrode, or attached to an optical element for use in combination.

The words “front”, “rear”, “top”, “bottom”, “above”, “below”, etc., if present, in the specification and claims are used for descriptive purposes and are not necessarily used to describe an invariant relative position. It should be understood that the words so used are interchangeable where appropriate, such that the embodiments of the present disclosure described herein, for example, are capable of operation in other orientations than those illustrated or otherwise described herein.

As used herein, the word “exemplary” means “used as an example, instance, or illustration” rather than serving as a “model” to be exactly copied. Any implementation described as an example herein is not necessarily to be construed as preferred or advantageous over other implementations. Moreover, the present disclosure is not limited by any stated or implied theory given in the above sections of technical field, background, summary, or detailed description.

As used herein, the term “substantially” is intended to include any minor variations due to design or manufacturing imperfections, device or element tolerances, environmental influences, and/or other factors. The term “substantially” also allows for deviations from a perfect or ideal situation due to parasitic effects, noise, and other practical considerations that may exist in actual implementations.

The terms “first”, “second” and the like may be used herein for reference purposes only and are not intended to be limiting. For example, the terms “first”, “second” and other such numerical terms referring to structures or elements do not imply a sequence or order unless the context clearly indicates otherwise.

It should also be understood that when the term “include/comprise” is used herein, it indicates the presence of the stated features, entities, steps, operations, units and/or components, but does not exclude the presence or addition of one or more other features, entities, steps, operations, units and/or components and/or combinations thereof.

As used herein, the term “and/or” includes any and every combinations of one or more of the associated listed items. The terms used herein are for the purpose of describing specific embodiments only and are not intended to limit the present disclosure. As used herein, the singular forms “a”, “an”, and “the” are also intended to include the plural forms, unless the context clearly indicates otherwise.

Those skilled in the art will appreciate that the boundaries between the above operations are merely illustrative. Multiple operations may be combined into a single operation, a single operation may be distributed in additional operations, and operations may be performed at least partially overlapping in time. Moreover, alternative embodiments may include multiple instances of a particular operation, and the operation order may be changed in other various embodiments. However, other modifications, variations, and replacements are also possible. Therefore, this specification and accompanying drawings should be considered illustrative, not restrictive.

Although some specific embodiments of the present disclosure have been described in detail by way of example, it should be understood by those skilled in the art that the above examples are for illustration only and are not intended to limit the scope of the present disclosure. The various embodiments disclosed herein may be combined in any manner without departing from the spirit and scope of the present disclosure. It should also be understood by those skilled in the art that various modifications may be made to the embodiments without departing from the scope and spirit of the present disclosure. The scope of the present disclosure is defined by the appended claims.

1. A flexible electrode apparatus for bonding with a SEEG electrode, comprising:
at least one wire electrode which is implantable and flexible, wherein each of the at least one wire electrode comprises:
a wire located between a first insulating layer and a second insulating layer of the flexible electrode; and
an electrode site located on the second insulating layer and electrically coupled to the wire via a through hole in the second insulating layer, wherein
the at least one wire electrode is configured to be affixed to the SEEG electrode and is in contact with a biological tissue after the SEEG electrode is implanted.

2. The flexible electrode apparatus according to claim 1, wherein:
the wire in each wire electrode comprises a plurality of wires located in a wire layer of the flexible electrode and spaced apart from each other, and
the electrode site in each wire electrode comprises a plurality of electrode sites each electrically coupled to one of the plurality of wires via a corresponding through hole in the second insulating layer.

3. The flexible electrode apparatus according to claim 1, further comprising:
a back-end portion comprising at least one back-end site,
wherein the at least one wire electrode each extends from the back-end portion; and
each back-end site is electrically coupled to a back-end circuit and one of wires via a through hole in the first insulating layer or the second insulating layer to achieve bidirectional signal transmission between the back-end circuit and an electrode site electrically coupled to the one of the wires.

4. The flexible electrode apparatus according to claim 1, wherein:
the wire electrode has a thickness of 300 nm to 200 μm.

5. The flexible electrode apparatus according to claim 1, further comprising:
a flexible separation layer, wherein the flexible separation layer is capable of being removed by a specific substance to separate a part of the flexible electrode and avoid damage to the flexible electrode.

6. The flexible electrode apparatus according to claim 5, wherein:
a material of the flexible separation layer is any one of nickel, chromium, or aluminum, or a combination thereof.

7. The flexible electrode apparatus according to claim 1, wherein:
a material of the first insulating layer and the second insulating layer is any one of polyimide, polydimethylsiloxane, parylene, epoxy resin, polyamide imide, polylactic acid, polylactic acid-glycolic acid copolymer, SU8 photoresist, silica gel, or silicone rubber, or a combination thereof.

8. The flexible electrode apparatus according to claim 1, wherein:
the first insulating layer and the second insulating layer have a thickness of 100 nm to 300 μm.

9. The flexible electrode apparatus according to claim 1, wherein:
the electrode site and the wire in each wire electrode comprise a conductive metal layer and an adhesion layer, respectively.

10. The flexible electrode apparatus according to claim 9, wherein:
a material of the conductive metal layer is any one of gold, platinum, iridium, tungsten, magnesium, molybdenum, platinum-iridium alloy, titanium alloy, graphite, carbon nanotubes, or PEDOT, or a combination thereof, and the conductive metal layer has a thickness of 5 nm to 200 μm; and
a material of the adhesion layer comprises chromium, tantalum, tantalum nitride, titanium or titanium nitride, and the adhesion layer has a thickness of 1 to 50 nm.

11. The flexible electrode apparatus according to claim 1, wherein:
the at least one wire electrode is affixed to a surface of the SEEG electrode in a form of attachment.

12. The flexible electrode apparatus according to claim 1, wherein:
the at least one wire electrode is affixed to a surface of the SEEG electrode by a mechanical structure.

13. The flexible electrode apparatus according to claim 12, wherein:
the mechanical structure comprises a gap formed by customizing a structure of the SEEG electrode, through which the flexible electrode can pass.

14. The flexible electrode apparatus according to claim 1, wherein:
the at least one wire electrode is attached to a surface of the SEEG electrode by a biodegradable material.

15. The flexible electrode apparatus according to claim 14, wherein:
the biodegradable material comprises any one of polyethylene glycol, polylactic acid, polylactic acid-glycolic acid copolymer, or silk protein, or a combination thereof.

16. The flexible electrode apparatus according to claim 1, wherein:
a material of the SEEG electrode is any one of platinum-iridium alloy, platinum, silver, or stainless steel, or a combination thereof, and the SEEG electrode has an inner diameter of 0.5 mm to 2 mm.

17. An implantable electrode apparatus, comprising:
a SEEG electrode and at least one wire electrode which is implantable and flexible,
wherein each of the at least one wire electrode comprises:
a wire located between a first insulating layer and a second insulating layer of the flexible electrode; and
an electrode site located on the second insulating layer and electrically coupled to the wire via a through hole in the second insulating layer,
wherein the at least one wire electrode is configured to be affixed to the SEEG electrode and is in contact with a biological tissue after the SEEG electrode is implanted.

18. A method for manufacturing a flexible electrode apparatus, the flexible electrode apparatus comprising the flexible electrode for bonding with a SEEG electrode according to claim 1, the method comprising:
forming a flexible separation layer over a substrate;
forming the first insulating layer, a wire layer, the second insulating layer, and an electrode site layer over the flexible separation layer in a layer-by-layer manner; and
removing the flexible separation layer to separate the flexible electrode from the substrate,
wherein before the electrode site layer is formed, a through hole is formed at a position corresponding to the electrode site in the second insulating layer by patterning.

19. A method for processing a flexible electrode apparatus, the flexible electrode apparatus comprising the flexible electrode for bonding with a SEEG electrode according to claim 1, the method comprising:
causing the SEEG electrode to be in contact with and attached to a root portion of the flexible electrode in a liquid;
adjusting an attaching angle, and slowly pulling an assembly of the SEEG electrode and the flexible electrode out of the liquid; and
baking the assembly to enhance adhesion between the SEEG electrode and the flexible electrode.
